(19) United States
(12) Patent Application Publication
(10) Pub. No.: US 2006/0258917 A1
(43) Pub. Date: Nov. 16, 2006

(54) Apparatus and Method of Use for Non-Invasive Analyte Measurement

(75) Inventors: John F. Burd, San Diego, CA (US); Paul Williams, San Diego, CA (US); William J. Sell, Petlaluma, CA (US)

Correspondence Address: PROCOPIO, CORY, HARGREAVES & SAVITCH LLP, 530 B STREET, SUITE 2100, SAN DIEGO, CA, 92101, US

(73) Assignee: OCULIR, INC., San Diego, CA (US)

(21) Appl. No.: 11/460,162

(22) Filed: Jul. 26, 2006

Related U.S. Application Data
(63) Continuation-in-part of application No. 11/122,472, filed on May 5, 2005.
(63) Continuation of application No. 10/824,214, filed on Apr. 14, 2004, now Pat. No. 6,975,892.

(51) Int. Cl. A61B 5/00; G06Q 99/00; G06F 17/00

(52) U.S. Cl. 600/309; 700/90; 705/1

(57) ABSTRACT

An apparatus and method of use for enabling a NAM device are provided. The NAM device is configured to authenticate its use each time a measurement is requested. The operation of the NAM device is governed by a refillable media card that tracks a predetermined number of authorized uses. The NAM device confirms that remaining authorized uses are available on the refillable media card. In addition to tracking the number of authorized uses of the NAM device, the media card may also store the results of the measurements for tracking by a tracking server. When the number of authorized uses on the media card is depleted, the media card may be refilled by completing a refill transaction over a wired or wireless communication network. Alternatively, the media card may be refilled at a kiosk station or a new media card may be obtained for use with the NAM device.

### RELATED APPLICATION

This application is a continuation-in-part application of U.S. patent application Ser. No. 11/122,472, filed May 5, 2005, which is a continuation application of U.S. patent application Ser. No. 10/824,214, filed Apr. 14, 2004, and claims the benefit of prior provisional application 60/513,396, filed on Oct. 21, 2003 under 35 U.S.C. 119(e). This application claims the benefit of these prior applications and these applications are incorporated by reference herein as though set forth in full.

### BACKGROUND

1. Field of the Invention

The present invention generally relates to mobile medical diagnostic measurement devices and more particularly relates to a media card authorization apparatus and its method of use.

2. Related Art

Diabetes remains one of the most serious and under-treated diseases facing the worldwide healthcare system. Diabetes is a chronic disease where the body fails to maintain normal levels of glucose in the bloodstream. It is now the fifth leading cause of death from disease in the U.S. today and accounts for about 15% of the entire healthcare budget. People with diabetes are classified into two groups: Type 1 (formerly known as “juvenile onset” or “insulin dependent” diabetes, that are required to take insulin to maintain life) and Type 2 (formerly known as “adult onset” or “non-insulin dependent,” that may require insulin but may sometimes be treated by diet and oral hypoglycemic drugs). In both cases, without dedicated and regular blood glucose measurement, all patients face the possibility of the complications of diabetes that include cardiovascular disease, kidney failure, blindness, amputation of limbs and premature death.

The number of cases of diabetes in the U.S. has jumped 40% in the last decade. This high rate of growth is believed to be due to a combination of genetic and lifestyle origins that appear to be a long-term trend, including obesity and poor diet. The American Diabetes Association (“ADA”) and others estimate that about 17 million Americans and over 150 million people worldwide have diabetes, and it is estimated that up to 40% of these people are currently undiagnosed [Diabetes Association, “Facts & Figures”].

Diabetes must be “controlled” in order to delay the onset of the disease complications. Therefore, it is essential for people with diabetes to measure their blood glucose levels several times per day in an attempt to keep their glucose levels within the normal range (80 to 126 mg/dL). These glucose measurements are used to determine the amount of insulin or alternative treatments necessary to bring the glucose level to within target limits. Self-Monitoring of Blood Glucose (“SMBG”) is an ongoing process repeated multiple times per day for the rest of the patient's lifetime.

All currently Food and Drug Administration (“FDA”) approved invasive or “less-invasive” (blood taken from the arm or other non-fingertip site) glucose monitoring products currently on the market require the drawing of blood in order to make a quantitative measurement of blood glucose. The ongoing and frequent measurement requirements (1 to possibly 10 times per day) presents all diabetic patients with pain, skin trauma, inconvenience, and infection risk resulting in a general reluctance to frequently perform the critical measurements necessary for selecting the appropriate insulin dose or other therapy.

These current product drawbacks have led to a poor rate of patient compliance. Among Type 1 diabetics, 39% measure their glucose levels less than once per day and 21% do not monitor their glucose at all. Among Type 2 diabetics who take insulin, only 26% monitor at least once per day and 47% do not monitor at all. Over 75% of non-insulin-taking Type 2 diabetics never monitor their glucose levels [Roper Starch Worldwide Survey]. Of 1,186 diabetics surveyed, 91% showed interest in a non-invasive glucose monitor. As such, there is both a tremendous interest and clinical need for a non-invasive glucose measurement device. A further need exists for systems and methods to track glucose measurements from a non-invasive glucose measurement device to ensure timely use and for monitoring the use of such a device to ensure compliance.

### SUMMARY

Accordingly, the present invention provides systems and methods for enabling the use of a non-invasive analyte measurement (“NAM”) device through a refillable media card that allows the non-invasive analyte measurement device to operate regardless of location. The NAM device is configured to measure one or more analyte levels of a user (e.g., the concentration, presence, and/or absence of one or more analytes). The ability to operate the NAM device may be governed by a media card that tracks a predetermined number of authorized uses. In addition to tracking the number of authorized uses of the NAM device, the media card may also store the results of the measurements for tracking by a tracking server. When the number of authorized uses on the media card is depleted, the media card may be refilled by completing a refill transaction over a wired or wireless communication network. Alternatively, the media card may be refilled at a kiosk station or a new media card may be obtained.

Other features and advantages of the present invention will become more readily apparent to those of ordinary skill in the art after reviewing the following detailed description and accompanying drawings.

### BRIEF DESCRIPTION OF THE DRAWINGS

The details of the present invention, both as to its structure and operation, may be gleaned in part by study of the accompanying drawings, in which like reference numerals refer to like parts, and in which:

FIG. 1 is a block diagram illustrating an example non-invasive analyte measurement device in operation according to an embodiment of the present invention;

FIG. 2 is a block diagram illustrating an non-invasive analyte measurement device and an example media card for use with the non-invasive analyte measurement device according to an embodiment of the present invention;

FIG. 3 is a network diagram illustrating an example system for refilling a media card for use with a non-invasive analyte measurement device according to an embodiment of the present invention;

FIG. 4 is a block diagram illustrating an example media card for use with a non-invasive analyte measurement device according to an embodiment of the present invention;

FIG. 5 is a flow diagram illustrating an example process for use of a media card with a predetermined number of authorized uses for a non-invasive analyte measurement device according to an embodiment of the present invention;

FIG. 6 is a block diagram illustrating an example wireless communication device that may be used in connection with various embodiments described herein; and

FIG. 7 is a block diagram illustrating an example computer system that may be used in connection with various embodiments described herein.

### DETAILED DESCRIPTION

Certain embodiments as disclosed herein provide for a media card for use with a non-invasive analyte measurement (“NAM”) device. In one embodiment, the media card contains a certain number of pre-authorized uses of the NAM device and authenticates the continued use of the NAM device. When the number of pre-authorized uses is depleted or near depleted, the media card can be refilled or replaced to allow continued pre-authorized operation of the NAM device. For example, a network or kiosk transaction can replenish the number of pre-authorized uses on the media card or a new card can be purchased at a pharmacy or other convenient location.

After reading this description it will become apparent to one skilled in the art how to implement the invention in various alternative embodiments and alternative applications. However, although various embodiments of the present invention will be described herein, it is understood that these embodiments are presented by way of example only, and not limitation. As such, this detailed description of various alternative embodiments should not be construed to limit the scope or breadth of the present invention as set forth in the appended claims.

Additionally, in the context of this application, the term “analyte” as used herein describes any particular substance or chemical constituent to be measured. Analyte may also include any substance in the tissue of a subject, in a biological fluid (for example, blood, interstitial fluid, cerebral spinal fluid, lymph fluid or urine), or is present in air that was in contact with or exhaled by a subject, which demonstrates an electromagnetic radiation signature, for example, infrared. Analyte may also include any substance which is foreign to or not normally present in the body of the subject. Analytes can include naturally occurring substances, artificial substances, metabolites, and/or reaction products. In some embodiments, the analyte for measurement by the devices and methods described herein is glucose. However, other analytes are contemplated as well, including, but not limited to, metabolic compounds or substances, carbohydrates such as sugars including glucose, proteins, glycated proteins, fructosamine, hemoglobin Alc, peptides, amino acids, fats, fatty acids, triglycerides, polysaccharides, alcohols including ethanol, toxins, hormones, vitamins, bacteria-related substances, fungus-related substances, virus-related substances, parasite-related substances, pharmaceutical or non-pharmaceutical compounds, substances, pro-drugs or drugs, and any precursor, metabolite, degradation product or surrogate marker of any of the foregoing. Other analytes are contemplated as well, including, but not limited, to acarboxyprothrombin; acylcarnitine; adenine phosphoribosyl transferase; adenosine deaminase; albumin; alpha-fetoprotein; amino acid profiles (arginine (Krebs cycle), histidine/urocanic acid, homocysteine, phenylalanine/tyrosine, tryptophan); andrenostenedione; antipyrine; arabinitol enantiomers; arginase; benzoylecgonine (cocaine); biotinidase; biopterin; c-reactive protein; carnitine; carnosinase; CD4; ceruloplasmin; chenodeoxycholic acid; chloroquine; cholesterol; cholinesterase; conjugated 1-hydroxy-cholic acid; cortisol; creatine kinase; creatine kinase MM isoenzyme; cyclosporin A; d-penicillamine; de-ethylchloroquine; dehydroepiandrosterone sulfate; nucleic acids (deoxyribonucleic acids and ribonucleic acids including native and variant sequences related to acetylator polymorphism, alcohol dehydrogenase, alpha 1-antitrypsin, cystic fibrosis, Down's syndrome, Duchenne/Becker muscular dystrophy, glucose-6-phosphate dehydrogenase, hemoglobin A, hemoglobin S, hemoglobin C, hemoglobin D, hemoglobin E, hemoglobin F, D-Punjab, beta-thalassemia, hepatitis B virus, HCMV, HIV-1, HTLV-1, Leber hereditary optic neuropathy, MCAD, PKU, Plasmodium vivax, sexual differentiation, 21-hydroxylase); 21-deoxycortisol; desbutylhalofantrine; dihydropteridine reductase; diptheria/tetanus antitoxin; erythrocyte arginase; erythrocyte protoporphyrin; esterase D; fatty acids/acylglycines; free -human chorionic gonadotropin; free erythrocyte porphyrin; free thyroxine (FT4); free tri-iodothyronine (FT3); fumarylacetoacetase; galactose/gal-1-phosphate; galactose-1-phosphate uridyltransferase; gentamicin; glucose-6-phosphate dehydrogenase; glutathione; glutathione perioxidase; glycocholic acid; glycosylated hemoglobin; halofantrine; hemoglobin variants; hexosaminidase A; human erythrocyte carbonic anhydrase I; 17-alpha-hydroxyprogesterone; hypoxanthine phosphoribosyl transferase; immunoreactive trypsin; lactate; lead; lipoproteins ((a), B/A-1, ); lysozyme; mefloquine; netilmicin; phenobarbitone; phenytoin; phytanic/pristanic acid; progesterone; prolactin; prolidase; purine nucleoside phosphorylase; quinine; reverse tri-iodothyronine (rT3); selenium; serum pancreatic lipase; sissomicin; somatomedin C; specific antibodies (adenovirus, anti-nuclear antibody, anti-zeta antibody, arbovirus, Aujeszky's disease virus, dengue virus, Dracunculus medinensis, Echinococcus granulosus, Entamoeba histolytica, enterovirus, Giardia duodenalisa, Helicobacter pylori, hepatitis B virus, herpes virus, HIV-1, IgE (atopic disease), influenza virus, Leishmania donovani, leptospira, measles/mumps/rubella, Mycobacterium leprae, Mycoplasma pneumoniae, Myoglobin, Onchocerca volvulus, parainfluenza virus, Plasmodium falciparum, poliovirus, Pseudomonas aeruginosa, respiratory syncytial virus, rickettsia (scrub typhus), Schistosoma mansoni, Toxoplasma gondii, Trepenoma pallidium, Trypanosoma cruzi/rangeli, vesicular stomatis virus, Wuchereria bancrofti, yellow fever virus); specific antigens (hepatitis B virus, HIV-1); neurotransmitters (such as glutamate, GABA, dopamine, serotonin), opioid neurotransmitters (such as endorphins, and dynorphins), neurokinins (such as substance P); succinylacetone; sulfadoxine; theophylline; thyrotropin (TSH); thyroxine (T4); thyroxine-binding globulin; trace elements; transferrin; UDP-galactose-4-epimerase; urea; prokaryotic and eukaryotic cell-surface antigens; peptidoglycans; lipopolysaccharide; uroporphyrinogen I synthase; vitamin A; white blood cells; and zinc protoporphyrin. Salts naturally occurring in blood or interstitial fluids can also constitute analytes in certain embodiments. The analyte can be naturally present in the biological fluid, for example, a metabolic product, an antigen, an antibody, and the like. Alternatively, the analyte can be introduced into the body, for example, a contrast agent for imaging, a radioisotope, a chemical agent, a fluorocarbon-based synthetic blood, or pharmaceutical composition, including but not limited to insulin; ethanol; cannabis (marijuana, tetrahydrocannabinol, hashish); inhalants (nitrous oxide, amyl nitrite, butyl nitrite, chlorohydrocarbons, hydrocarbons); cocaine (crack cocaine); stimulants (amphetamines, methamphetamines, Ritalin, Cylert, Preludin, Didrex, PreState, Voranil, Sandrex, Plegine); depressants (barbiturates, methaqualone, tranquilizers such as Valium, Librium, Miltown, Serax, Equanil, Tranxene); tricyclic antidepressants, benzodiazepines, acetaminophen (paracetamol, APAP), aspirin, methadone, hallucinogens (phencyclidine, lysergic acid, mescaline, peyote, psilocybin); narcotics (heroin, codeine, morphine, opium, meperidine, Percocet, Percodan, Tussionex, Fentanyl, Darvon, Talwin, Lomotil); designer drugs (analogs of fentanyl, meperidine, amphetamines, methamphetamines, and phencyclidine, for example, Ecstasy); anabolic steroids; and nicotine. The metabolic products of drugs and pharmaceutical compositions are also contemplated analytes. Analytes such as neurochemicals and other chemicals generated within the body can also be analyzed, such as, for example, ascorbic acid, uric acid, dopamine, noradrenaline, 3-methoxytyramine (3MT), 3,4-dihydroxyphenylacetic acid (DOPAC), homovanillic acid (HVA), 5-hydroxytryptamine (5HT), and 5-hydroxyindoleacetic acid (5HIAA).

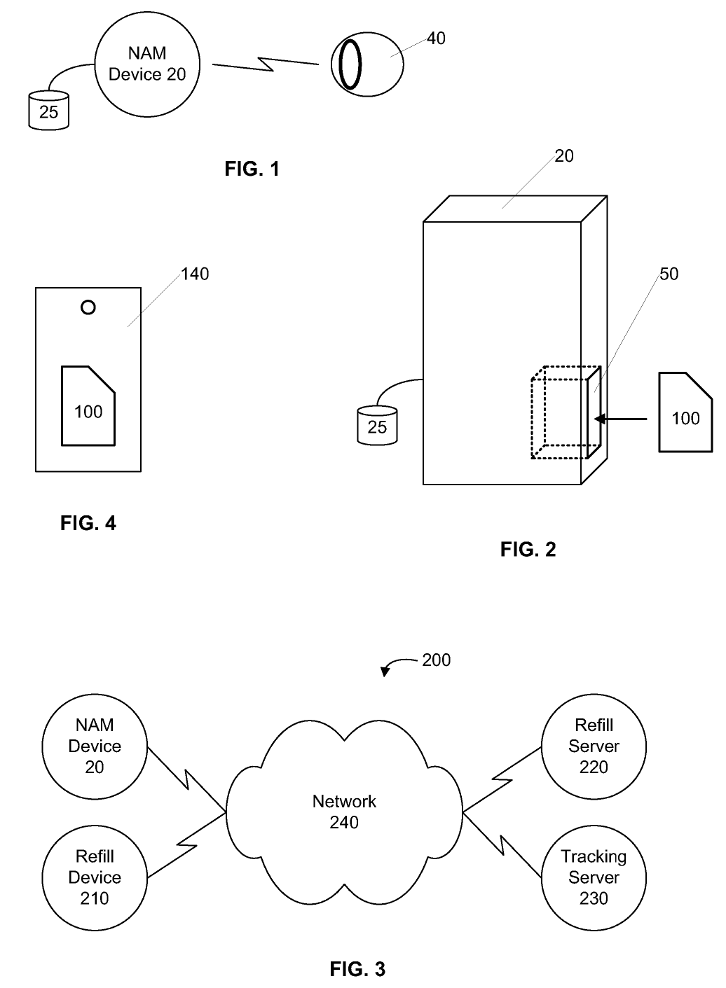

FIG. 1 is a block diagram illustrating an example wireless NAM device 20 in operation according to an embodiment of the present invention. In the illustrated embodiment, the NAM device 20 interrogates a body surface of the subject, for example the eye 40. Advantageously, the interrogation can be accomplished using electrogmagnetic signals, and more advantageously, infrared (“IR”) signals, such that the measurement is taken non-invasively. As a result of the interrogation, the NAM device 20 measures one or more analyte levels (e.g., the concentration, presence, and/or absence of one or more analytes) for the subject and the measured concentrations can be stored in the data storage area 25. An example non-invasive analyte measurement device is described in U.S. patent application Ser. No. 11/122,472, which is incorporated by reference herein as though set forth in full. Measurements can be taken periodically by the NAM device 20 such that the data for multiple interrogations can be stored in the data storage area 25.

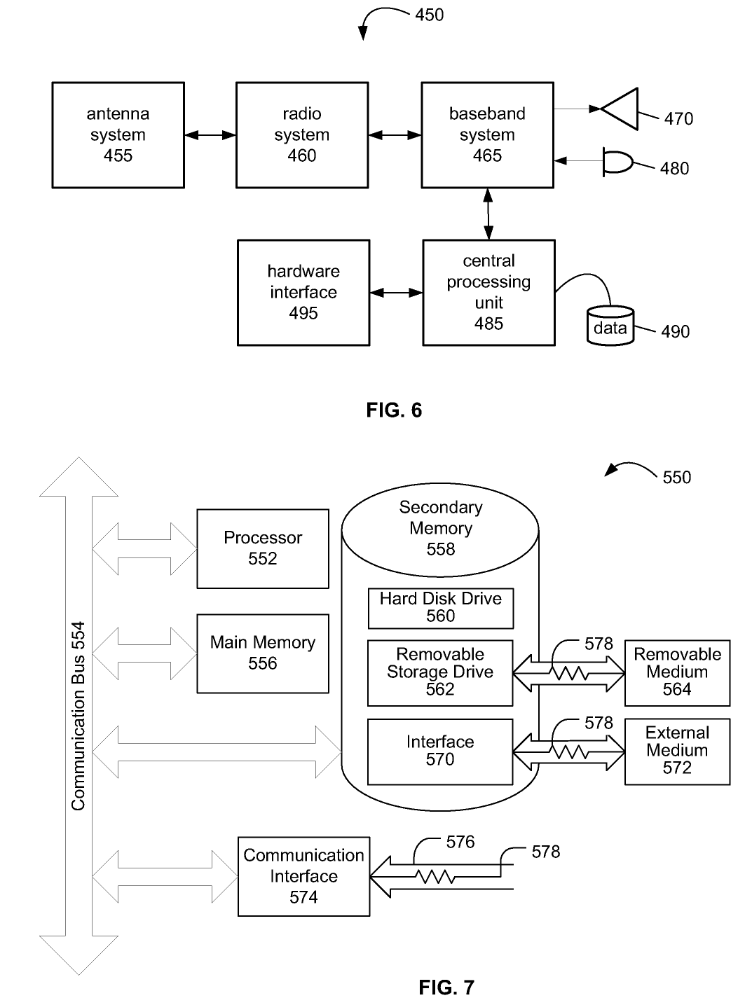

The NAM device 20 is a non-invasive analyte measurement device and can be integrated into any of a variety of types of wired or wireless communication devices including a personal digital assistant (“PDA”), cellular telephone, handheld gaming device, personal computer, laptop computer, specific purpose device, general purpose device, or other device that is capable of use as or modification for use as a non-invasive measurement device. A general purpose wireless communication device is described later with respect to FIG. 6 and a general purpose computer device is described later with respect to FIG. 7. As will be understood by those having skill in the art, each of these types of devices are suitable for modification and use as the NAM device 20.

The data storage area 25 can be any sort of internal or external, fixed or removable memory device and may include both persistent and volatile memories. The function of the data storage area 35 is to maintain data for long term storage and also to provide efficient and fast access to instructions for applications or modules that are executed by the NAM device 20.

FIG. 2 is a block diagram illustrating an example media card 100 for use with a NAM device 20 according to an embodiment of the present invention. In one or more embodiments, the media card 100 is any of a variety of types of media cards, for example, but not by way of limitation, compact flash, memory stick, micro drive, multimedia card, smart media card, picture card, card disk, hyper drive, spy disk, walk key, jump drive, or the like. The function of the media card 100 is to store data and information related to the use of the NAM 20 and any sort of media device capable of achieving this function may be employed.

As shown in the illustrated embodiment, the card 100 is inserted into the NAM 20 for integrated use, for example by inserting the card 100 into a media slot 50 that is configured to receive a media card 100 or other memory storage device. Advantageously, the card 100 contains a certain number of pre-authorized uses for the NAM device 20 and is also configured in combination with the NAM device 20 to store information related to the various measurements taken by the NAM device 20.

FIG. 3 is a network diagram illustrating an example system 200 for refilling a media card 100 for use with a NAM device 20 according to an embodiment of the present invention. In the illustrated embodiment, the system 200 comprises NAM device 20 and refill device 210. These devices are communicatively coupled with a refill server 220 and a tracking server 230 over a network 240. In one or more embodiments, the system 200 includes more or fewer NAM devices 20, refill devices 210, refill servers 220, and/or tracking servers 230.

In one embodiment, the NAM device 20 is configured for network communication and can therefore communication directly with the refill server 220 to replenish the number of authorized uses stored on a media card 100 being used with the NAM device 20. Such communication may be a simple authorization from the NAM device 20 to charge an account associated with the particular NAM device 20 and in return replenishes the number of authorized uses on the media card 100. Alternatively, such a communication is an interactive session that walks through a refill transaction that also results in replenishing the number of authorized uses on the media card 100.

The network 240 is any of a variety of network types and topologies and any combination of such types and topologies. Network 240 is one or more of a telephone network, a data network, a wired network, a wireless network or any combination of these. For example, in one or more embodiments, the network 240 comprises a plurality of networks including private, public, circuit switched, packet switched, personal area networks (“PAN”), local area networks (“LAN”), wide area networks (“WAN”), metropolitan area networks (“MAN”), or any combination of the these. In one or more embodiments, network 240 includes the particular combination of networks ubiquitously known as the Internet.

The refill device 210 is any of a variety of computing devices and platforms that are capable of communication with NAM device 20 and/or the refill server 220 and/or the tracking server 230 over the network 240. In one embodiment, the refill device 210 is resident at a public kiosk and includes a media card reader capable of reading from and writing to a media card 100 being used with the NAM device 20. The refill device 210 is also configured to communicate with the refill server 220 over the network 240 and to facilitate a transaction that provides the media card 100 with additional pre-authorized uses. In one or more embodiments, the refill device 210 is a dongle that attaches to a general purpose computer or the refill device 210 is integrated with a kiosk device. Advantageously, the refill device 210 may be located in a convenient place such as a local pharmacy, Internet cafe, integrated with a public telephone, ATM machine, or other location/device.

The refill server 220 is any of a variety of computing devices and platforms that are capable of communication with NAM device 20 or refill device 210 over the network 240. The refill server 220 is configured to process a transaction with a remote device (e.g., NAM device 20 or refill device 210) and replenish the number of pre-authorized uses stored on a memory card. Advantageously, this may be accomplished over network 240 such that a single refill server 220 may contemporaneously process requests for a significant number of NAM devices 20 and refill devices 210.

The tracking server 230 can be any of a variety of computing devices and platforms that are capable of communication with NAM device 20 or refill device 210 over the network 240. In one embodiment, the tracking server 230 is integrated with the refill server 220. The tracking server 230 is configured to track information related to the use of a NAM device 20, for example information including the measurements taken by the NAM device 20 and the number of measurements taken by the NAM device 20. The tracking server 230 is optional but advantageous in that it can help users of the NAM devices 20 to spot trends over time as related to an individual's analyte measurements (e.g., the concentration, presence, and/or absence of one or more analytes) at different times of day and other beneficial metrics may also be tracked and reported by the tracking server 230.

FIG. 4 is a block diagram illustrating an example media card 100 for use with a NAM device 20 according to an embodiment of the present invention. In the illustrated embodiment, the media card 100 is provided in a packaging 140 that advantageously allows for distribution of the media card 100 through retail outlets and vending machines. For example, in one or more embodiments, media card 100 is sold at a local pharmacy. In one or more embodiments, the media card 100 is sold with a predetermined number of authorized uses such as, but not limited to 100 uses, 200 uses, 500 uses, and 1000 uses. In one or more embodiments, different media cards 100 come with various numbers of authorized uses, depending on the need of the individual user. In one embodiment, a NAM device 20 is integrated with a kiosk device that allows any person from the general public to use the device to measure the individual's analyte levels (e.g., the concentration, presence, and/or absence of one or more analytes). In such an embodiment, the purveyor of the kiosk device may purchase a media card 100 with 1000 authorized uses so that the kiosk needs infrequent servicing to replenish the number of authorized uses.

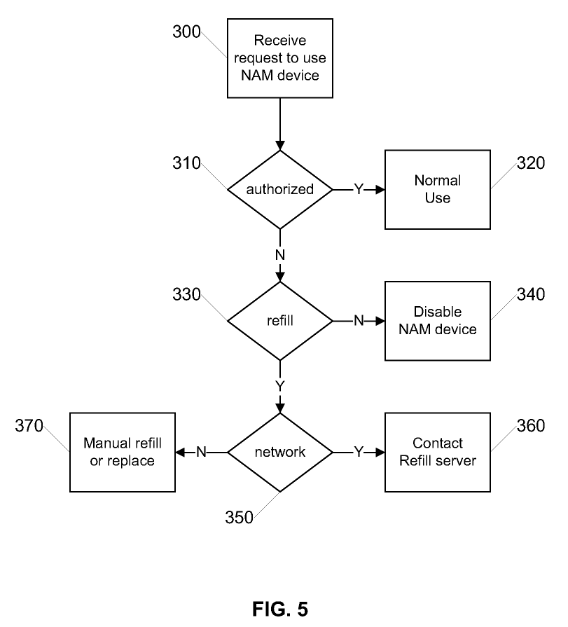

FIG. 5 is a flow diagram illustrating an example process for use of a media card 100 with a predetermined number of authorized uses for a NAM device 20 according to an embodiment of the present invention. The illustrated process may be carried out by a NAM device 20 such as that previously described with respect to FIGS. 1 and 3. Initially, in step 300 the NAM device 20 receives a request for use. The request can come from a user of the NAM device 20 by depressing a button or speaking a command or by some other means. For example, in an exemplary embodiment, the user holds the device in proximity to a body surface such as the eye and press a button for one or more analyte measurements to be taken (e.g., the concentration, presence, and/or absence of one or more analytes).

In step 310, the NAM device 20 determines if the NAM device 20 is authorized for use. For example, in one embodiment, the NAM device 20 is leased with a certain number of pre-authorized uses. Advantageously, the number of pre-authorized uses is identified on a media card device and updated by the NAM device 20 after each use of the NAM device 20. Accordingly, in step 310 the NAM device 20 determines if there are additional authorized uses remaining and if there are, the NAM device 20 proceeds to normal use in step 320.

If, however, there are no remaining authorized uses, the NAM device 20 next determines if the user wants to refill the number of authorized uses, as shown in step 330. In one embodiment, the NAM device 20 is configured so that additional authorized uses are automatically obtained without intervention by the user. Advantageously, the NAM device 20 is configured to obtain the additional authorized uses prior to the number of uses being completely depleted so that there is no inconvenience to the user with respect to the need for obtaining additional authorized uses.

In step 330 if the NAM device 20 determines that the user does not wish to obtain more authorized uses, in the step 340 the NAM device 20 disables the measurement functionality until such authorized uses are obtained. Advantageously, other functions of the NAM device 20 are still usable, for example if the NAM device 20 is combined with a cell phone or PDA or the like.

In step 330 if the NAM device 20 determines that the user does wish to obtain more authorized uses, in step 350 the NAM device 20 determines if it is able to replenish the authorized uses via an available network connection. If a network connection is available, the NAM device 20 contacts a refill server 220, as shown in step 360. Advantageously, as part of the network communication, the NAM device 20 provides additional measurement related information to the refill server 220 or a tracking server 230.

If no network connection is available, as determined in step 350, then the user manually replenishes the authorized uses at a kiosk or other refill device, as shown in step 370. Alternatively, the user provides the NAM device 20 with a new or different media card 100 that includes additional authorized uses. Thus, it can be advantageous for a user to carry an additional media card 100 with authorized uses stored on it so that the user is able to take a measurement, for example, when the primary media card 100 is depleted and the user is out of range of any network that would provide the NAM device 20 with access to a refill server 220.

FIG. 6 is a block diagram illustrating an example wireless communication device 450 used in connection with one or more embodiments described herein. For example, in one or more embodiments, the wireless communication device 450 is used in conjunction with the NAM device 20 or the refill device 210 previously described with respect to FIG. 3. However, in alternative embodiments, other wireless communication devices and/or architectures are used, as will be clear to those skilled in the art.

In the illustrated embodiment, wireless communication device 450 comprises an antenna system 455, a radio system 460, a baseband system 465, a speaker 470, a microphone 480, a central processing unit (“CPU”) 485, a data storage area 490, and a hardware interface 495. In the wireless communication device 450, radio frequency (“RF”) signals are transmitted and received over the air by the antenna system 455 under the management of the radio system 460.

In one embodiment, the antenna system 455 comprises one or more antennae and one or more multiplexors (not shown) that perform a switching function to provide the antenna system 455 with transmit and receive signal paths. In the receive path, received RF signals can be coupled from a multiplexor to a low noise amplifier (not shown) that amplifies the received RF signal and sends the amplified signal to the radio system 460.

In alternative embodiments, the radio system 460 comprises one or more radios that are configured to communication over various frequencies. In one embodiment, the radio system 460 combines a demodulator (not shown) and modulator (not shown) in one integrated circuit (“IC”). Alternatively, the demodulator and modulator are separate components. In the incoming path, the demodulator strips away the RF carrier signal leaving a baseband receive audio signal, which is sent from the radio system 460 to the baseband system 465.

If the received signal contains audio information, then baseband system 465 decodes the signal and converts it to an analog signal. Then the signal is amplified and sent to the speaker 470. The baseband system 465 also receives analog audio signals from the microphone 480. These analog audio signals are converted to digital signals and encoded by the baseband system 465. The baseband system 465 also codes the digital signals for transmission and generates a baseband transmit audio signal that is routed to the modulator portion of the radio system 460. The modulator mixes the baseband transmit audio signal with an RF carrier signal generating an RF transmit signal that is routed to the antenna system and may pass through a power amplifier (not shown). The power amplifier amplifies the RF transmit signal and routes it to the antenna system 455 where the signal is switched to the antenna port for transmission.

The baseband system 465 is also communicatively coupled with the central processing unit 485. The central processing unit 485 has access to a data storage area 490. The central processing unit 485 is preferably configured to execute instructions (i.e., computer programs or software) that can be stored in the data storage area 490. Computer programs can also be received from the baseband processor 465 and stored in the data storage area 490 or executed upon receipt. Such computer programs, when executed, enable the wireless communication device 450 to perform the various functions of the present invention as previously described. For example, data storage area 490 includes various software modules (not shown) that facilitate the operation of the various functions of the invention.

In this description, the term “computer readable medium” is used to refer to any media used to provide executable instructions (e.g., software and computer programs) to the wireless communication device 450 for execution by the central processing unit 485. Examples of these media include the data storage area 490, microphone 470 (via the baseband system 465), antenna system 455 (also via the baseband system 465), and hardware interface 495. These computer readable mediums are means for providing executable code, programming instructions, and software to the wireless communication device 450. The executable code, programming instructions, and software, when executed by the central processing unit 485, preferably cause the central processing unit 485 to perform the inventive features and functions previously described herein.

The central processing unit 485 is also preferably configured to receive notifications from the hardware interface 495 when new devices are detected by the hardware interface. Hardware interface 495 can be a combination electromechanical detector with controlling software that communicates with the CPU 485 and interacts with new devices. The hardware interface 495 may be a firewire port, a USB port, a Bluetooth or infrared wireless unit, or any of a variety of wired or wireless access mechanisms. Examples of hardware linkable with the device 450 include data storage devices, computing devices, headphones, microphones, and the like.

FIG. 7 is a block diagram illustrating an exemplary computer system 550 used in connection with one or more embodiments described herein. For example, in one embodiment, the computer system 550 is used in conjunction with the refill server 220 and/or the tracking server 230 previously described with respect to FIG. 3. However, in alternative embodiments, other computer systems and/or architectures are used, as will be clear to those skilled in the art.

The computer system 550 preferably includes one or more processors, such as processor 552. Additional processors may be provided, such as an auxiliary processor to manage input/output, an auxiliary processor to perform floating point mathematical operations, a special-purpose microprocessor having an architecture suitable for fast execution of signal processing algorithms (e.g., digital signal processor), a slave processor subordinate to the main processing system (e.g., back-end processor), an additional microprocessor or controller for dual or multiple processor systems, or a coprocessor. Such auxiliary processors are discrete processors or integrated with the processor 552.

The processor 552 is preferably connected to a communication bus 554. The communication bus 554 includes a data channel for facilitating information transfer between storage and other peripheral components of the computer system 550. The communication bus 554 provides a set of signals used for communication with the processor 552, including a data bus, address bus, and control bus (not shown). The communication bus 554 comprises any standard or non-standard bus architecture such as, for example, bus architectures compliant with industry standard architecture (“ISA”), extended industry standard architecture (“EISA”), Micro Channel Architecture (“MCA”), peripheral component interconnect (“PCI”) local bus, or standards promulgated by the Institute of Electrical and Electronics Engineers (“IEEE”) including IEEE 488 general-purpose interface bus (“GPIB”), IEEE 696/S-100, and the like.

Computer system 550 preferably includes a main memory 556 and, in one or more embodiments, includes a secondary memory 558. The main memory 556 provides storage of instructions and data for programs executing on the processor 552. The main memory 556 is typically semiconductor-based memory such as dynamic random access memory (“DRAM”) and/or static random access memory (“SRAM”). Other semiconductor-based memory types include, for example, synchronous dynamic random access memory (“SDRAM”), Rambus dynamic random access memory (“RDRAM”), ferroelectric random access memory (“FRAM”), and the like, including read only memory (“ROM”).

In one or more embodiments, the secondary memory 558 includes a hard disk drive 560 and/or a removable storage drive 562, for example a floppy disk drive, a magnetic tape drive, a compact disc (“CD”) drive, a digital versatile disc (“DVD”) drive, etc. The removable storage drive 562 reads from and/or writes to a removable storage medium 564 in a well-known manner. Removable storage medium 564 may be, for example, a floppy disk, magnetic tape, CD, DVD, etc.

The removable storage medium 564 is preferably a computer readable medium having stored thereon computer executable code (i.e., software) and/or data. The computer software or data stored on the removable storage medium 564 is read into the computer system 550 as electrical communication signals 578.

In alternative embodiments, secondary memory 558 includes other similar means for allowing computer programs or other data or instructions to be loaded into the computer system 550. Such means include, for example, an external storage medium 572 and an interface 570. Examples of external storage medium 572 include an external hard disk drive or an external optical drive, or and external magneto-optical drive.

Other examples of secondary memory 558 include semiconductor-based memory such as programmable read-only memory (“PROM”), erasable programmable read-only memory (“EPROM”), electrically erasable read-only memory (“EEPROM”), or flash memory (block oriented memory similar to EEPROM). Also included are any other removable storage units 572 and interfaces 570, which allow software and data to be transferred from the removable storage unit 572 to the computer system 550.

In one or more embodiments, computer system 550 includes a communication interface 574. The communication interface 574 allows software and data to be transferred between computer system 550 and external devices (e.g. printers), networks, or information sources. For example, computer software or executable code are transferred to computer system 550 from a network server via communication interface 574. Examples of communication interface 574 include a modem, a network interface card (“NIC”), a communications port, a PCMCIA slot and card, an infrared interface, and an IEEE 1394 fire-wire, just to name a few.

Communication interface 574 preferably implements industry promulgated protocol standards, such as Ethernet IEEE 802 standards, Fiber Channel, digital subscriber line (“DSL”), asynchronous digital subscriber line (“ADSL”), frame relay, asynchronous transfer mode (“ATM”), integrated digital services network (“ISDN”), personal communications services (“PCS”), transmission control protocol/Internet protocol (“TCP/IP”), serial line Internet protocol/point to point protocol (“SLIP/PPP”), and so on, but may also implement customized or non-standard interface protocols as well.

Software and data transferred via communication interface 574 are generally in the form of electrical communication signals 578. These signals 578 are preferably provided to communication interface 574 via a communication channel 576. Communication channel 576 carries signals 578 and can be implemented using a variety of wired or wireless communication means including wire or cable, fiber optics, conventional phone line, cellular phone link, wireless data communication link, radio frequency (RF) link, or infrared link, just to name a few.

Computer executable code (i.e., computer programs or software) is stored in the main memory 556 and/or the secondary memory 558. In one or more embodiments, computer programs are received via communication interface 574 and stored in the main memory 556 and/or the secondary memory 558. Such computer programs, when executed, enable the computer system 550 to perform the various functions of the present invention as previously described.

In this description, the term “computer readable medium” is used to refer to any media used to provide computer executable code (e.g., software and computer programs) to the computer system 550. Examples of these media include main memory 556, secondary memory 558 (including hard disk drive 560, removable storage medium 564, and external storage medium 572), and any peripheral device communicatively coupled with communication interface 574 (including a network information server or other network device). These computer readable mediums are means for providing executable code, programming instructions, and software to the computer system 550.

In an embodiment that is implemented using software, the software is stored on a computer readable medium and loaded into computer system 550 by way of removable storage drive 562, interface 570, or communication interface 574. In such an embodiment, the software is loaded into the computer system 550 in the form of electrical communication signals 578. The software, when executed by the processor 552, preferably causes the processor 552 to perform the inventive features and functions previously described herein.

Another embodiment is implemented primarily in hardware using, for example, components such as application specific integrated circuits (“ASICs”), or field programmable gate arrays (“FPGAs”). Implementation of a hardware state machine capable of performing the functions described herein will also be apparent to those skilled in the relevant art. One or more further embodiments are implemented using a combination of both hardware and software.

Furthermore, in one or more embodiments, the various illustrative logical blocks, modules, circuits, and method steps described in connection with the above described figures and the embodiments disclosed herein are implemented as electronic hardware, computer software, or combinations of both. To clearly illustrate this interchangeability of hardware and software, various illustrative components, blocks, modules, circuits, and steps have been described above generally in terms of their functionality. Whether such functionality is implemented as hardware or software depends upon the particular application and design constraints imposed on the overall system. Skilled persons can implement the described functionality in varying ways for each particular application, but such implementation decisions should not be interpreted as causing a departure from the scope of the invention. In addition, the grouping of functions within a module, block, circuit or step is for ease of description. Specific functions or steps are movable from one module, block or circuit to another without departing from the invention.

Moreover, in one or more embodiments, the various illustrative logical blocks, modules, and methods described herein are implemented or performed with a general purpose processor, a digital signal processor (“DSP”), an ASIC, FPGA or other programmable logic device, discrete gate or transistor logic, discrete hardware components, or any combination thereof designed to perform the functions described herein. In one or more embodiments, a general-purpose processor is a microprocessor. Alternatively, the processor is any processor, controller, microcontroller, or state machine. In one or more embodiments, a processor is implemented as a combination of computing devices, for example, a combination of a DSP and a microprocessor, a plurality of microprocessors, one or more microprocessors in conjunction with a DSP core, or any other such configuration.

Additionally, in one or more embodiments, the steps of a method or algorithm described in connection with the embodiments disclosed herein are embodied directly in hardware, in a software module executed by a processor, or in a combination of the two. A software module resides in RAM memory, flash memory, ROM memory, EPROM memory, EEPROM memory, registers, hard disk, a removable disk, a CD-ROM, or any other form of storage medium including a network storage medium. An exemplary storage medium is coupled to the processor such the processor reads information from, and writes information to, the storage medium. In the alternative, the storage medium is integral to the processor. In one or more embodiments, the processor and the storage medium reside in an ASIC.

The above description of the disclosed embodiments is provided to enable any person skilled in the art to make or use the invention. Various modifications to these embodiments will be readily apparent to those skilled in the art, and the generic principles described herein can be applied to other embodiments without departing from the spirit or scope of the invention. Thus, it is to be understood that the description and drawings presented herein represent a presently preferred embodiment of the invention and are therefore representative of the subject matter which is broadly contemplated by the present invention. It is further understood that the scope of the present invention fully encompasses other embodiments that may become obvious to those skilled in the art and that the scope of the present invention is accordingly limited by nothing other than the appended claims.

1. A non-invasive analyte measurement device comprising:
means for non-invasive interrogation of analyte measurement information;
a removable data storage apparatus comprising a number of authorized uses for the non-invasive analyte measurement device;
a control program configured to receive an instruction to take a measurement with said means for non-invasive interrogation and read the number of authorized uses from the removable data storage apparatus to determine if the instruction is to be executed.

2. The device of claim 1, further comprising a network communication means configured to obtain additional authorized uses from a refill server via a communication network and add said number of additional authorized uses to said number of authorized uses in the removable data storage apparatus and store the sum as the number of authorized uses in the removable data storage apparatus.

3. The device of claim 2, wherein the network communication means is configured for communication over a wireless communication network.

4. The device of claim 2, wherein the network communication means is configured for communication over a wired communication network.

5. The device of claim 2, wherein the communication network is a data network.

6. The device of claim 2, wherein the communication network is a voice network.

7. The device of claim 1, wherein the means for non-invasive interrogation of analyte measurement information is configured to obtain analyte measurement information from a body surface.

8. A method for authorized use of a non-invasive analyte measurement device, comprising:
receiving a request at a non-invasive analyte measurement device to obtain analyte measurement information;
reading a removable data storage apparatus to determine a number of authorized uses for the non-invasive analyte measurement device;
confirming that the number of authorized uses is greater than zero;
obtaining the requested analyte measurement information; and
decrementing the number of authorized uses; and
storing the decremented number of authorized uses as the number of authorized uses on the removable data storage apparatus.

9. The method of claim 8, wherein the confirming step comprises:
accessing a refill server via a communication network;
requesting additional authorized uses for the non-invasive analyte measurement device;
receiving the requested additional authorized uses; and
updating the number of authorized uses on the removable data storage apparatus.

10. The method of claim 9, wherein the communication network is a wired communication network.

11. The method of claim 9, wherein the communication network is a wireless communication network.

12. The method of claim 9, wherein the communication network is a voice network.

13. The method of claim 9, wherein the communication network is a data network.

14. The method of claim 8, wherein the confirming step comprises:
removing the removable data storage apparatus from the non-invasive analyte measurement device;
inserting the removable data storage apparatus into a refill device;
updating the number of authorized uses on the removable data storage apparatus; and
returning the removable data storage apparatus to the non-invasive analyte measurement device.

15. The method of claim 14, wherein the refill device is communicatively coupled with a refill server via a communication network.

16. The method of claim 15, wherein the communication network is a wired communication network.

17. The method of claim 15, wherein the communication network is a wireless communication network.

18. The method of claim 15, wherein the communication network is a voice network.

19. The method of claim 15, wherein the communication network is a data network.

20. The method of claim 8, wherein the confirming step comprises:
removing the removable data storage apparatus from the non-invasive analyte measurement device;
inserting a new removable data storage apparatus into the non-invasive analyte measurement device, wherein the new removable data storage apparatus comprises a number of authorized uses greater than zero.
